(19) United States
(12) Patent Application Publication
(10) Pub. No.: US 2011/0269720 A1
(43) Pub. Date: Nov. 3, 2011

(54) MINOCYCLINE AND RIFAMPIN MICROPARTICLES

(75) Inventors: Christopher M. Hobot, Tonka Bay, MN (US); Randall V. Sparer, Andover, MN (US); Mark E. Hughes, North Branch, MN (US); William Ferris, Minneapolis, MN (US)

(73) Assignee: MEDTRONIC, INC., Minneapolis, MN (US)

(21) Appl. No.: 12/768,913

(22) Filed: Apr. 28, 2010

(51) Int. Cl. A61K 31/65; A61P 31/04

(52) U.S. Cl. 514/154

(57) ABSTRACT

Methods and kits for treating infection associated with implantation of a medical device use of minocycline and rifampin microparticles. The microparticles, in a suitable medium, can be injected in a patient in proximity to the device. The drugs may be configured to be released from the polymer matrix in a controlled manner by manipulation of the properties of the polymer forming the microparticle. By injecting the drugs in the form of microparticles, the drugs can be distributed in a manner so that the entire pocket is protected without affecting device function. The microparticles can be produced aseptically without affecting the manufacturing of the device.

### FIELD

The present disclosure relates generally to systems and methods for treating infection due to implantation of a medical device; particularly to the use of microparticles containing minocycline and rifampin in treating such infections.

### BACKGROUND

Antimicrobial loaded polymers are currently being used to decrease infection rates in medical devices such as bladder and central venous catheters, penile prostheses and chronic wound dressings. Significant reduction in clinical infection rates has been demonstrated using controlled release of the drug combination of rifampin and minocycline in biocompatible silicones. Reducing infection in this manner becomes more difficult when the device is more complex; such as in the case of implantable pulse generators or infusion pumps which have an outer shell that is composed mainly of titanium, and thus cannot be impregnated with antimicrobial drugs.

Attempts to coat the devices with the drug combination in a suitable polymer have drawbacks as well. For example, the sterilization procedure most commonly used for the more complex devices is ethylene oxide treatment, which may not be compatible with the antimicrobial agent(s). In addition, coating large portions of the device can also affect device function. Under these constraints only a coating that is applied aseptically after the device has been sterilized and only covers a portion of the device is plausible. The manufacturing and application of such a device may prove difficult.

### SUMMARY

This disclosure, among other things, describes the use of minocycline and rifampin microparticles for treating (e.g., preventing) an infection associated with implantation of a medical device. The microparticles, in a suitable medium, can be injected in a patient in proximity to the device. The drugs may be configured to be released from the polymer matrix in a controlled manner by manipulation of the properties of the polymer forming the microparticle. By injecting the drugs in the form of microparticles, the drugs can be distributed in a manner so that the entire pocket is protected without affecting device function. The microparticles can be produced aseptically, or terminally sterilized separate from the device, without affecting the manufacturing of the device.

A given microparticle may include both minocycline and rifampin. Alternatively, or in addition, minocycline microparticles and rifampin microparticles may be produced separately. By forming minocycline microparticles and rifampin microparticles separately, incompatibilities between minocycline and rifampin may be reduced and processes that favor compatibility for each drug may be employed. In some embodiments, minocycline microparticles and rifampin microparticles are kept separate until just prior to injecting into a patient, which should further serve to reduce compatibility and potential stability issues.

In various embodiments, a kit is described. The kit includes a first container containing minocycline microparticles and a second container containing rifampin microparticles. The kit may further include a third container in which the minocycline and rifampin microparticles may be placed prior to injecting in the patient. The third container may include markings that indicate a level to which the minocycline and rifampin microparticles may be added to achieve a desired ratio of minocycline and rifampin, and may include a marking indicating a level to which a solution may be added to suspend the microparticles prior to injection into the patient. In some embodiments, the first or second containers may contain such markings, and the rifampin microparticles may be added to the first container or the minocycline microparticles may be added to the second container. In many embodiments, the first and second containers contain an amount of minocycline and rifampin microparticles appropriate for a single use. That is, the entire contents of each of the first and second containers may be infused into a patient in one use.

In numerous embodiments, a microparticle comprising a polymer and minocycline and rifampin is described. The polymer may be biodegradable. A plurality of the microparticles may be placed in proximity to a medical device implanted in a patient to prevent infection associated with the implantation of the device.

Advantages of one or more embodiments of the methods, devices, systems and kits described herein will be apparent to those of skilled in the art upon reading the following detailed description.

### BRIEF DESCRIPTION OF THE DRAWINGS

The accompanying drawings, which are incorporated into and form a part of the specification, illustrate several embodiments of the present disclosure and, together with the description, serve to explain the principles of the disclosure. The drawings are only for the purpose of illustrating embodiments of the disclosure and are not to be construed as limiting the disclosure.

FIG. 1 is a schematic drawing of an environment of an infusion system implanted in a patient.

FIG. 2 is a schematic drawing of an environment of an electrical signal generator system implanted in a patient

FIGS. 3A-D are a schematic drawings of environments of medical devices implanted in patients.

FIG. 4 is a flow diagram illustrating an overview of an embodiment of a method of treating an infection associated with implantation of a medical device.

FIGS. 5-13 are schematic diagrams of kits or various components of kits.

The schematic drawings presented herein are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description, reference is made to the accompanying drawings that form a part hereof, and in which are shown by way of illustration several embodiments of devices, systems and methods. It is to be understood that other embodiments are contemplated and may be made without departing from the scope or spirit of the present disclosure. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise.

As used in this specification and the appended claims, the term “or” is generally employed in its sense including “and/or” unless the content clearly dictates otherwise.

As used herein, “have”, “having”, “include”, “including”, “comprise”, “comprising” or the like are used in their open ended sense, and generally mean “including, but not limited to.”

As used herein, the terms “treat”, “therapy”, and the like are meant to include methods to alleviate, slow the progression, prevent, attenuate, or cure the treated disease.

Reference herein to a particular therapeutic agent includes pharmaceutically acceptable salts, polymorphs, and hydrates thereof.

As used herein, “microparticle” means a particle having an average diametric dimension of less than 500 micrometers. For example, a microparticle may have an average diametric dimension of between about 2 and about 100 micrometers. Microparticles include particles having an average diametric dimension of less than 1 micrometers. That is microparticles include nanoparticles for the purposes of the present disclosure. In various embodiments, microparticles are microspheres. Microparticles are typically polymeric microparticles. Accordingly, a “minocycline microparticle” is a polymeric microparticle that includes minocycline. Similarly, a “rifampin microparticle” is a polymeric microparticle that includes rifampin. The minocycline or rifampin may be contained within, attached (covalently or noncovalently), or otherwise associated with the microparticle.

As used herein, an event that occurs “during a procedure for implanting a medical device” occurs at any time associated with the implant procedure. For example, the event may occur while the patient is being prepared for surgery; while the patient is in the operating room, such as just before, concurrently, or following implantation of the device; or the like.

The present disclosure, among other things, describes the use of minocycline and rifampin microparticles for treating (e.g., preventing) an infection associated with implantation of a medical device. Such microparticles may be used in connection with implantation of any medical device, such as a catheter, a lead, an implantable infusion device, an implantable electrical signal generator (e.g., a cardiac defibrillator, a pacemaker, a neurostimulator, a gastric stimulator, a cochlear implant), or the like.

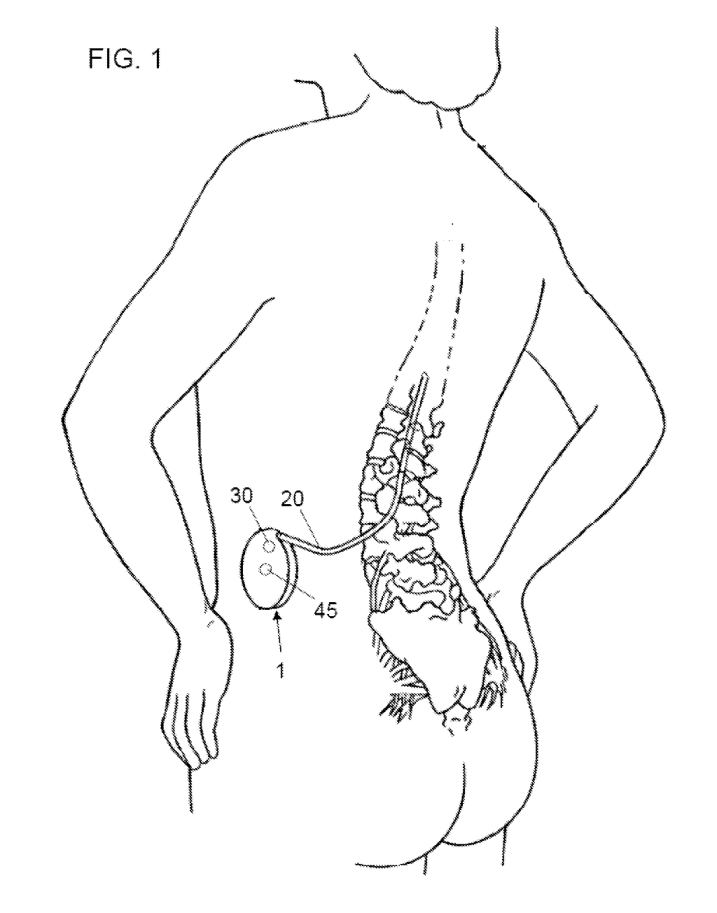

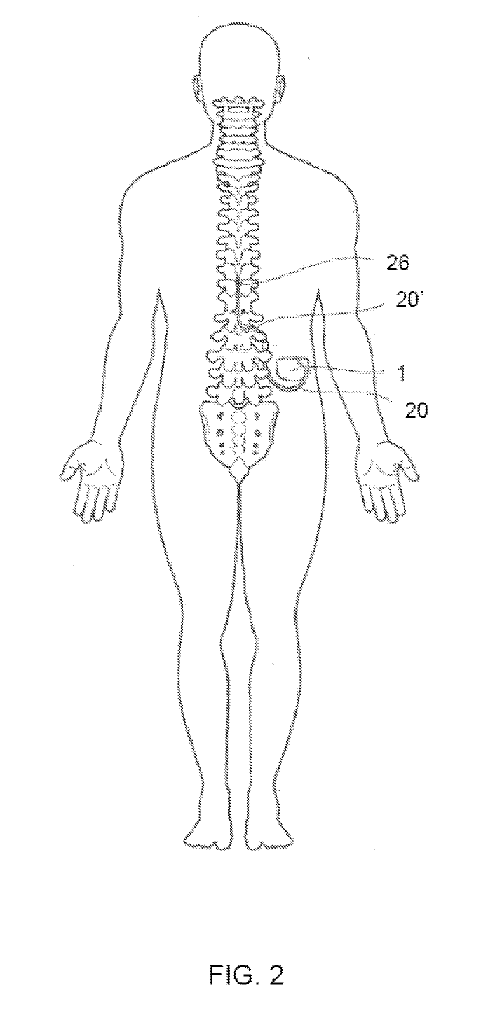

Referring to FIGS. 1 and 2, general illustrative environments for implanted active therapy delivering medical devices 1 and associated devices 20 are shown. In the depicted embodiments, active medical device 1 is subcutaneously implanted in an abdominal region of a patient. A distal portion of associated device 20 is intrathecally inserted into the patient's spinal canal through a lumbar puncture and advanced rostrally to a desired location (FIG. 1) or epidurally placed along a suitable location of spinal cord (FIG. 2). Proximal end of associated device 20 is tunneled subcutaneously to location of active device 1, where it may be connected to active device 1. While distal portion of associated device 20 is shown in FIGS. 1 and 2 as being located in or on spinal cord, it will be understood that associated device 20 may be placed at any location in patient for which it is desirable to administer therapy generated or delivered by active medical device 1. The process of implanting the active medical device 1 or the associated medical device 20, which often includes opening a subcutaneous pocket for implantation of the active device 1 and tunneling of the associated device 20, provides an opportunity for the introduction of an infectious agent and infection.

In the embodiment shown in FIG. 1, active implantable device 1 is an infusion device, and associated device 20 is a catheter. Catheter 20 is typically a flexible tube with a lumen running from the proximal end of catheter 20 to one or more delivery regions that are typically located at the distal portion of catheter 20. Proximal portion of catheter 20 is connected to infusion device 20. Distal portion of catheter 20 is positioned at a target location in the patient to deliver fluid containing therapeutic agent from infusion device 1 to patient through a delivery region of catheter 20. Infusion device 1, such as Medtronic Inc.'s SynchroMed™ 11 implantable programmable pump system, includes a reservoir (not shown) for housing a therapeutic substance and a refill port 45 in fluid communication with reservoir. The reservoir may be refilled by percutaneously inserting a needle (not shown) into patient such that needle enters refill port 45, and fluid containing therapeutic substance may be delivered into reservoir from needle via refill port 45. Infusion device 1 shown in FIG. 1 also includes a catheter access port 30 that is in fluid communication with the catheter 20. Fluid may be injected into or withdrawn from patient through catheter 20 via catheter access port 30 by percutaneously inserting a needle into access port 30. Each entry of needle across patient's skin to gain access refill port 45 or access port 30 results in the possibility of infection in proximity to the active medical device 1.

In the embodiment shown in FIG. 2, active implantable device 1 is an electrical signal generator, such as Medtronic Inc.'s Restore™ Advanced implantable neurostimulator, and associated devices 20, 20′ are a lead extension 20 and lead 20′. Lead 20′ includes one or more electrical contacts (not shown) on its proximal end portion and one or more electrodes on its distal end portion 26. The contacts and electrodes are electrically coupled via wires running through lead 20′. Electrical signals generated by the signal generator 1 may be delivered to lead 20 through the contacts and then to the patient through the electrodes. As shown in FIG. 2, lead 20′ may be connected to signal generator 1 through a lead extension 20. Extension 20 includes one or more contacts at the proximal and distal end portions that are electrically coupled through wires running through extension 20. Of course it will be understood that with some systems lead 20′ may be directly connected to electrical signal generator 1 without use of a lead extension 20. It will be further understood that more than one lead 20′ or lead extension 20 may be employed per signal generator 1.

While FIGS. 1 and 2 depict systems including infusion devices and catheters and electrical signal generators and leads, it will be understood that the teachings described herein may be applicable to virtually any known or future developed implantable medical device.

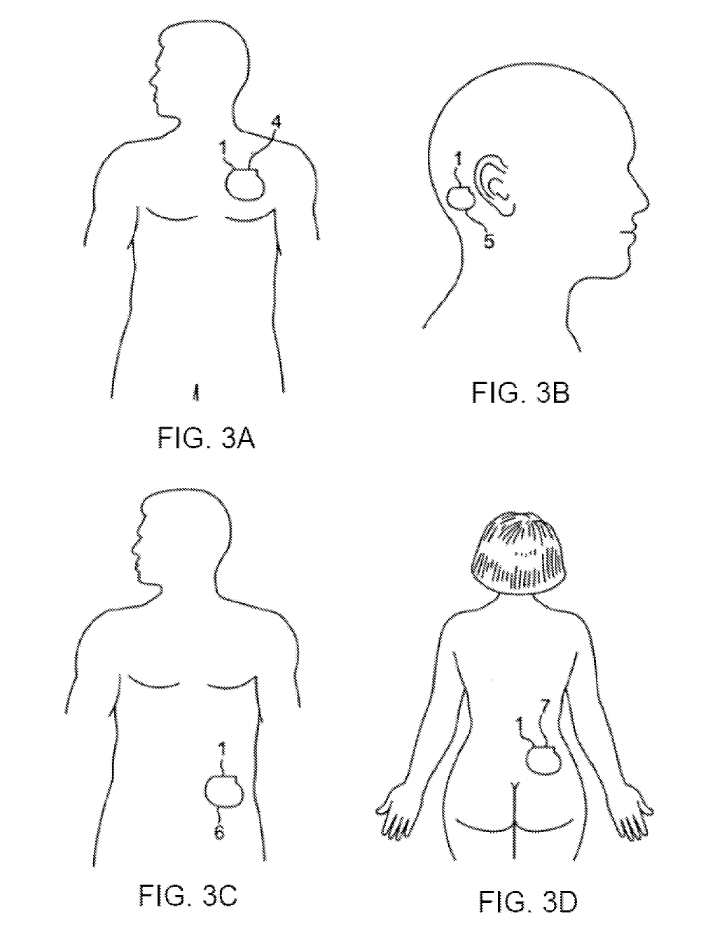

Referring to FIG. 3, alternative locations for implanting a medical device 1 are shown. As depicted in FIG. 3A, device 1 may be implanted in the pectoral region 7 of a patient. Alternatively, device 1 may be implanted in the head of a patient, more specifically behind the patient's ear (FIG. 3B), in the patient's abdomen (FIG. 3C) or in the patient's lower back or buttocks (FIG. 3D). Of course, device 1 may be placed in any medically acceptable location in patient. Regardless of the location of implantation of the device 1, the possibility of infection exists.

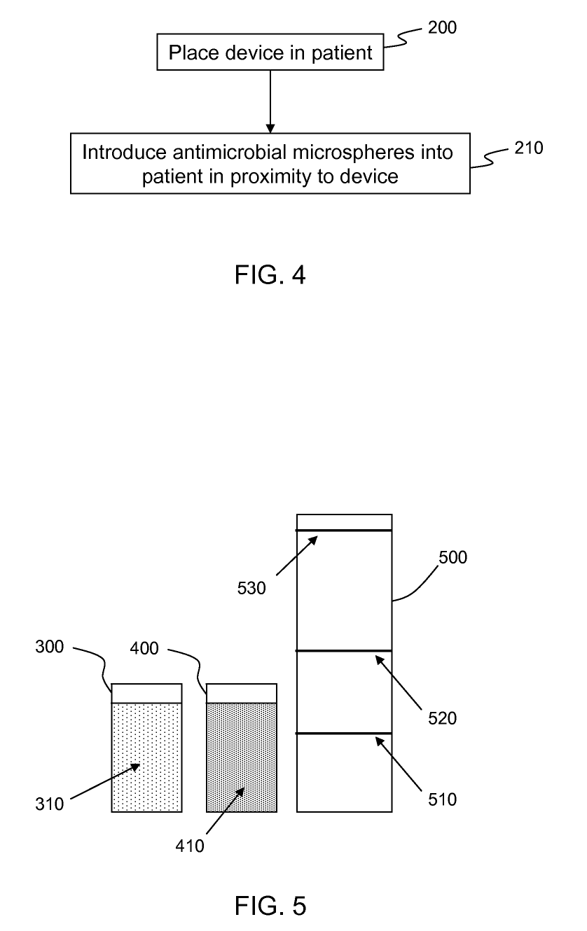

Referring now to FIG. 4, an overview of a method for treating an infection associated with implantation of a medical device in a patient is shown. The method includes placing an implantable medical device in a patient (200) and introducing antimicrobial microparticles into the patient in proximity to the implantable medical device (210). Any suitable antibiotic agent or agents may be associated with a microparticle for purposes of treating (e.g., preventing) and infection. Microparticles offer a variety of opportunities to control aspects of antibiotic administration. For example, the microparticle may offer protection or masking of the antibiotic agent or agents, may reduce the dissolution rate, and may facilitate the handling and storage of the agent. Microparticles may be tuned to accurately deliver quantities of antimicrobial agents by, for example, affecting the clearance kinetics, tissue distribution, metabolism, and cellular interactions of the antimicrobial agents.

Generally, the amount of antibiotic released from the microparticle should provide a local concentration (e.g. in proximity to the microparticle and medical device) greater than the minimum inhibitory concentration (MIC) of the agent or combination of agents against the infectious species responsible for the infection to be treated. Most often, infections associated with implantation of a medical device are due to one or more of Staphylococcus aureus, Staphlococcus epidermis, Pseudomonus auruginosa, and Candidia Sp. Accordingly, the microparticles may be configured to release antibiotic agents that produce a local level at or above the MIC for one or more of these agents. More preferably, the local level is at or above two or three times the MIC to reduce the likelihood of antimicrobial resistance. In addition, it may be desirable for the microparticle to release the antimicrobial agent at such levels for a sustained amount of time while the risk of development of infection remains high. As time passes following healing of the wound created during implantation of the device, the likelihood of infection decreases. Accordingly, it may be suitable for the microparticles to release the antibiotic agent(s) at, e.g., 2× MIC for 2 days, 1 week, or thirty days.

One of skill in the art will understand how to determine the MIC of antibiotics against various strains of infectious species; e.g., through routine experimentation or via an appropriate literature search. Any suitable method for forming drug-containing microparticles having desired properties to achieve desired release rates may be employed. For example, the methods for forming microparticles as described in U.S. Pat. No. 7,659,273; U.S. Pat. No. 7,282,220; U.S. Pat. No. 6,699,506; U.S. Pat. No. 6,428,477; U.S. Pat. No. 6,193,944; U.S. Pat. No. 5,871,851; U.S. Pat. No. 5,556,642; U.S. Pat. No. 5,023,081; U.S. Pat. No. 7,423,010; U.S. Pat. No. 6,676,972; U.S. Pat. No. 5,462,750; U.S. Pat. No. 5,078,994; U.S. Pat. No. 5,026,559; or the like may be employed.

Any anti-infective agent may be used in accordance with various embodiments of the disclosure. An anti-infective agent may be any agent effective at killing or inhibiting the growth of a microorganism or a population of microorganisms. For example, the anti-infective agent may be an antibiotic or an antiseptic.

Nonlimiting examples of classes of antibiotics that may be used include tetracyclines (e.g. minocycline), rifamycins (e.g. rifampin), macrolides (e.g. erythromycin), penicillins (e.g. nafcillin), cephalosporins (e.g. cefazolin), other beta-lactam antibiotics (e.g. imipenem, aztreonam), aminoglycosides (e.g. gentamicin), chloramphenicol, sulfonamides (e.g. sulfamethoxazole), glycopeptides (e.g. vancomycin), quinolones (e.g. ciprofloxacin), fusidic acid, trimethoprim, metronidazole, clindamycin, mupirocin, polyenes (e.g. amphotericin B), azoles (e.g. fluconazole) and beta-lactam inhibitors (e.g. sulbactam). Nonlimiting examples of specific antibiotics that may be used include minocycline, rifampin, erythromycin, nafcillin, cefazolin, imipenem, aztreonam, gentamicin, sulfamethoxazole, vancomycin, ciprofloxacin, trimethoprim, metronidazole, clindamycin, teicoplanin, mupirocin, azithromycin, clarithromycin, ofloxacin, lomefloxacin, norfloxacin, nalidixic acid, sparfloxacin, pefloxacin, amifloxacin, enoxacin, fleroxacin, temafloxacin, tosufloxacin, clinafloxacin, sulbactam, clavulanic acid, amphotericin B, fluconazole, itraconazole, ketoconazole, and nystatin. Other examples of antibiotics, such as those listed in Sakamoto et al., U.S. Pat. No. 4,642,104, which is herein incorporated by reference in its entirety, may also be used. One of ordinary skill in the art will recognize other antibiotics that may be used.

To enhance the likelihood that microorganisms will be killed or inhibited, it may be desirable to combine one or more antibiotic. It may also be desirable to combine one or more antibiotic with one or more antiseptic. It will be recognized by one of ordinary skill in the art that antimicrobial agents having different mechanisms of action or different spectrums of action may be most effective in achieving such an effect. In particular embodiments, a combination of rifampin and minocycline is used.

Nonlimiting examples of antiseptics include hexachlorophene, cationic bisiguanides (e.g. chlorhexidine, cyclohexidine), iodine and iodophores (e.g. povidone-iodine), para-chloro-meta-xylenol, triclosan, furan medical preparations (i.e. nitrofurantoin, nitrofurazone), methenamine, aldehydes (glutaraldehyde, formaldehyde), silver compounds (e.g., silver sulfadiazine) and alcohols. One of ordinary skill in the art will recognize other antiseptics. To enhance the likelihood that microbes will be killed or inhibited, it may be desirable to combine one or more antiseptics. It may also be desirable to combine one or more antiseptics with one or more antibiotics. It will be recognized by one of ordinary skill in the art that antimicrobial agents having different mechanisms of action or different spectrums of action may be most effective in achieving such an effect. In particular embodiments, a combination of chlorohexidine and silver sulfadiazine is used.

One or more antibiotic agents or antiseptic agents may be incorporated into a single microparticle. In some embodiments, a microparticle includes only one anti-infective agent so that incompatible agents may be processed and stored separately. For example, minocycline is often employed in its hydrochloride salt form, and rifampin is acid labile. While it may be desirable to incorporate both minocycline and rifampin into one microparticle, it may also be desirable to form separate microparticles, one incorporating minocycline-HCl and the other incorporating rifampin, so that rifampin is not kept in close proximity to the acidic minocycline.

The microparticles, regardless of the number of associated therapeutic agents may be formed from biostable or bioerodible components. It may be desirable for the microparticles to be formed from bioerodible or biodegradable particles so that the particles do not provide a long-term nuisance or discomfort to the patient. However, it is not expected that biostable microparticles would provide a great deal of discomfort.

Non-limiting examples of biodegradable or bioerodible polymers that may be employed in forming microparticles, include: poly(amides) such as poly(amino acids) and poly(peptides); poly(esters) such as poly(lactic acid), poly(glycolic acid), poly(lactic-co-glycolic acid), and poly(caprolactone); poly(anhydrides); poly(orthoesters); poly(carbonates); and chemical derivatives thereof (substitutions, additions of chemical groups, for example, alkyl, alkylene, hydroxylations, oxidations, and other modifications routinely made by those skilled in the art), fibrin, fibrinogen, cellulose, starch, collagen, and hyaluronic acid, copolymers and mixtures thereof. The properties and release profiles of these and other suitable polymers are known or readily identifiable. It will be understood that the anti-infective(s) agent may elute from an intact microparticle or may be released upon degradation of the microparticle.

Non-limiting examples of suitable biostable vehicles that may be used include organic polymers such as silicones, polyamines, polystyrene, polyurethane, acrylates, polysilanes, polysulfone, methoxysilanes, and the like. Other polymers that may be utilized include polyolefins, polyisobutylene and ethylene-alphaolefin copolymers; acrylic polymers and copolymers, ethylene-covinylacetate, polybutylmethacrylate; vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; polycarbonates; polyoxymethylenes; polyimides; polyethers; epoxy resins; polyurethanes; rayon; rayon-triacetate; cellulose; cellulose acetate, cellulose butyrate; cellulose acetate butyrate; cellophane; cellulose nitrate; cellulose propionate; cellulose ethers; carboxymethyl cellulose; polyphenyleneoxide; and polytetrafluoroethylene (PTFE).

In many embodiments, one or both of minocycline or rifampin are included in a microparticle. The microparticle may include a biodegradable polymer, such as poly(lactic-co-glycolic acid).

Referring now to FIGS. 5-12, various embodiments of kits, or components thereof, are depicted. The kits include a first container 300 containing minocycline microparticles 310 and second container 400 containing rifampin microparticles. The containers 300, 400 may take any suitable form and may be formed of any suitable material, including polymers, glass, or metals. The microparticles 310, 410 are preferably stored in the containers 300, 400 in a dry form. In various embodiments, the microparticles 310, 410 are vacuum packed in the containers 300, 400, and the containers are sealed. In some embodiments, the containers 300, 400 contain hinged or threaded lids (not shown).

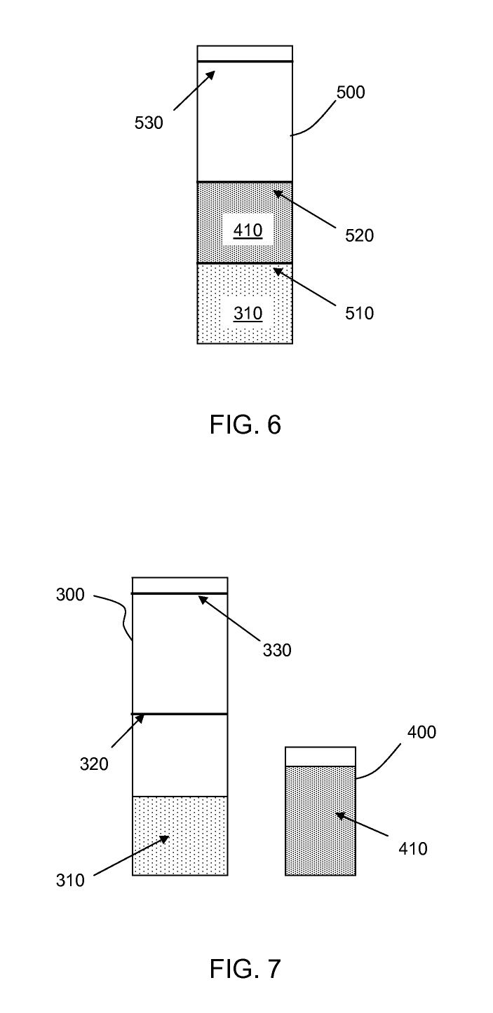

In the embodiments depicted in FIGS. 5-6, the kit further includes a third container 500. The third container 500 in the depicted embodiments includes a first marking 520, a second marking 520, and a third marking 530. The first marking 510 indicates a level to which one of the minocycline microparticles 310 or the rifampin microparticles 410 (minocycline microparticles 310 in the embodiment depicted in FIG. 6) should be placed in the container 500 to achieve a desired amount of the microparticles for delivery to the patient. The second marking 520 indicates a level to which the other of the minocycline microparticles 310 or the rifampin microparticles 410 (rifampin microparticles 410 in the embodiment depicted in FIG. 6) should be placed in the container 500 to achieve a desired amount and ratio of minocycline microparticles 310 to rifampin microparticles 410 for delivery to the patient in proximity to the implantable device. As depicted, the third container may contain a third marking 530 indicating the level to which a solution may be added to suspend the microparticles 310, 410 in a desired volume for purposes of infusing into the patient. Any suitable solution (not depicted) for suspending and infusing may be employed. Typically, the solution includes water (i.e., is aqueous). For example, the solution may be water, saline, buffered saline, or the like. The solution may be pre-sterilized, housed in a container (not shown in FIGS. 5-6), and included in the kit. The solution may contain, or be free from, preservatives or other pharmaceutically acceptable excipients.

In some embodiments, the third container may include no markings or one or more of the depicted markings 510, 520, 530, depending on the nature of the contents of the kit. For example, if the first container 300 includes a pre-determined amount of minocycline microparticles 310 for a single use, the first marking 510 on the third container 500 may be omitted. However, if the kit included an amount of minocycline microparticles 310 in excess of that for a single use, it may be desirable to include the first marking 510 on the third container 500. Similarly, if the kit included a pre-determined amount of rifampin microparticles 410 for a single use, the second marking 520 on the third container 500 may be omitted, or if the kit included a pre-determined amount of solution for a single use, the third marking 530 on the third container 500 may be omitted.

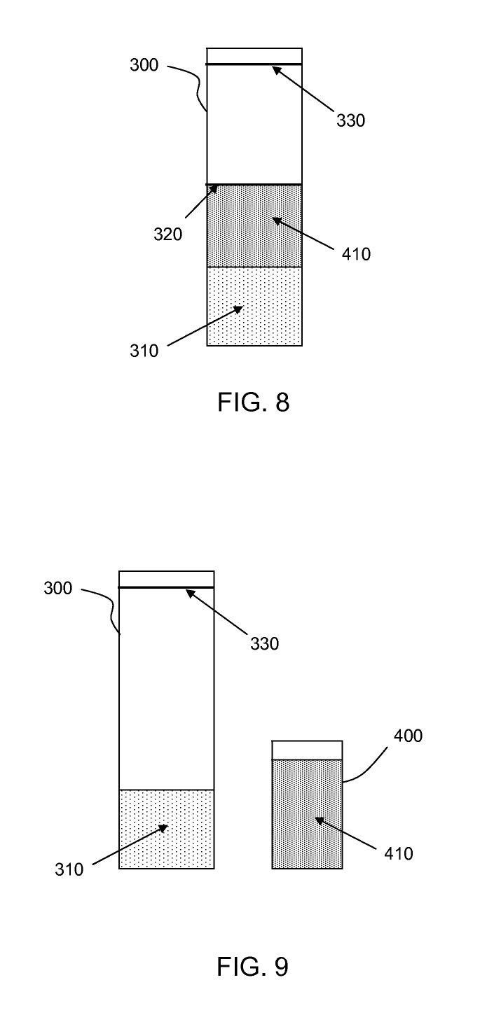

Referring now to FIG. 7, the first container 300 contains minocycline microparticles 310 in an amount suitable for a single use and has sufficient empty space to add rifampin microparticles in a desired amount and to add a suitable solvent or solution for suspending the microparticles. The first container 300 includes markings 320, 330. The first marking 320 indicates a level to which rifampin microparticles 410 from the second container 400 may be added to achieve a desired ratio of minocycline microparticles 310 to rifampin microparticles 410 (see FIG. 8). The third marking 330 indicates a level to which a solution for suspending the microparticles may be added, after the rifampin microparticles are added. Of course it will be understood that the second container 400 containing the rifampin microparticles 410 may include markings and have sufficient volume for addition of minocycline microparticles or a solution.

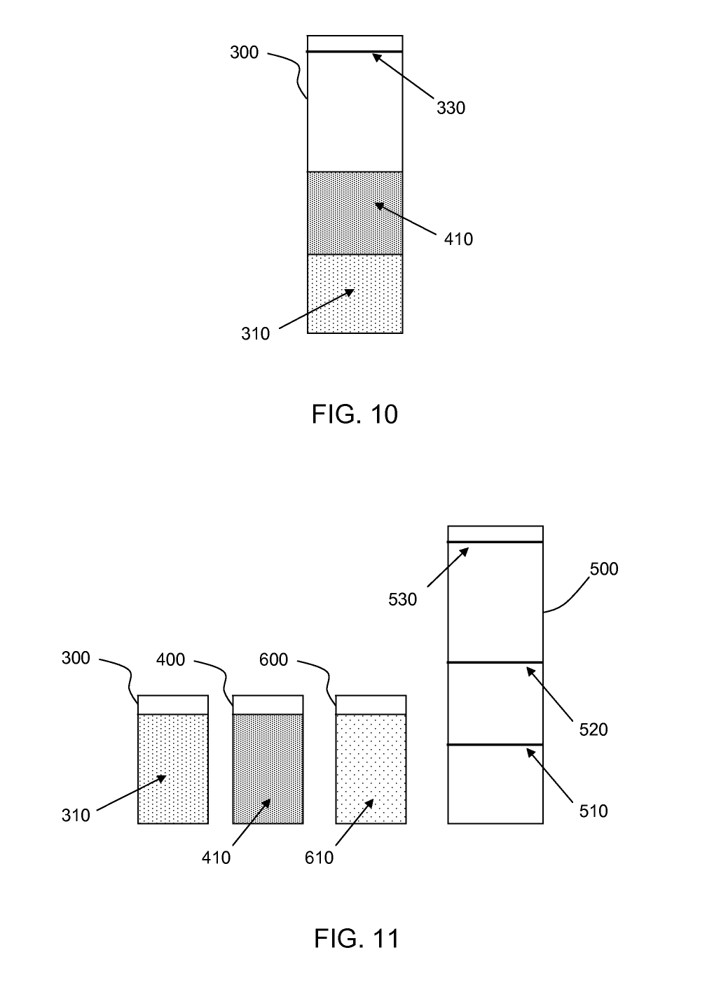

Referring now to FIGS. 9-10, the first container 300 contains an amount of minocycline microparticles 310 suitable for a single use. The second container 400 contains an amount of rifampin microparticles 410 suitable for a single use. The rifampin microparticles 410 may be added to the first container 300 just prior to use (see FIG. 10) and a solution may be then added to a level indicated by marking 330. Of course it will be understood that the second container 400 containing the rifampin microparticles 410 may include markings and have sufficient volume for addition of minocycline microparticles or a solution.

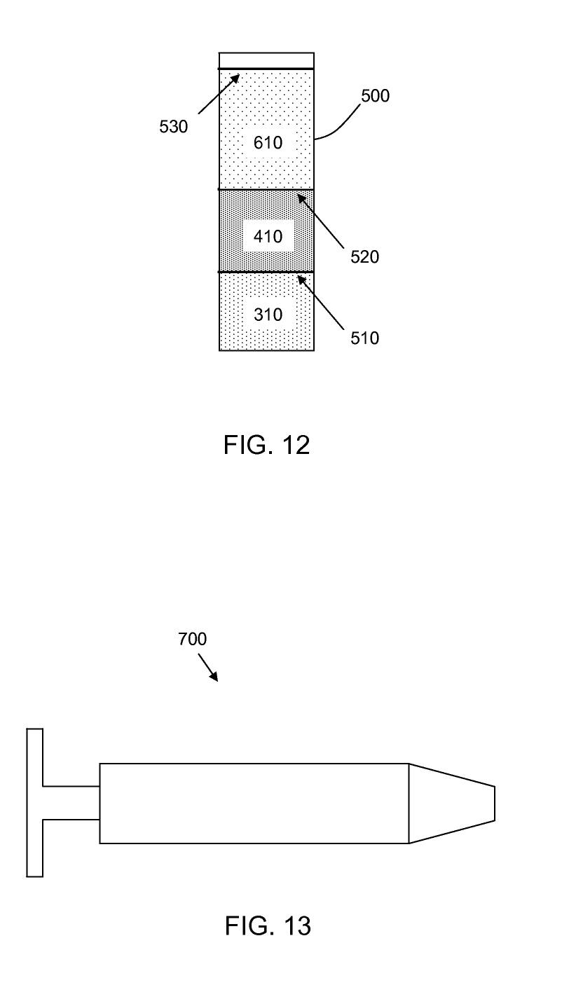

Referring now to FIGS. 11-12, the depicted kit includes a first container 300 containing minocycline microparticles 310, a second container 400 containing rifampin microparticles 410, a third container 500 having markings 510, 520, 530 and a fourth container 600 (a solvent container) containing a solution 610 for suspending the minocycline and rifampin microparticles. The minocycline microparticles 310 may be added to the third container 500 until reaching a level of marking 510. The rifampin microparticles 410 may be added to the third container 500 until reaching a level of marking 520, and the solution 610 may then be added until reaching a level of marking 530 to achieve a desired amount and ratio of minocycline microparticles, rifampin microparticles, and solution for delivering to a patient in proximity to an implantable medical device. Of course, the markings may be placed the first 300, second 400, or fourth 600 container and the container having the markings would have sufficient volume for the addition of desired amounts of the other components. If one or more of the component (microparticles or solution) are included in their respective containers in single use amounts, then the markings may be omitted, as discussed above with regard to FIGS. 5-10.

While the discussion above with regard to FIGS. 5-12 relates to mixing the minocycline and rifampin microparticles prior to delivery to a patient. It will be understood that the minocycline and rifampin microparticles may be administered separately.

It will also be understood that the volume of solution, as well as the amount of the microparticles (and thus the amount of antibiotic) may vary depending to the device implanted, the expected surgical pocket size, and the like. Preferably, the infused microparticles surround the implantable device in the surgical pocket.

The microspheres may be delivered to the patient in any suitable manner. For example, a syringe 700 (see e.g., FIG. 13) may be used to withdraw suspended microparticles from a container and to inject or infuse the microparticles into a patient. The syringe or other suitable device may be included in a kit, along with containers as described above.

Those skilled in the art will recognize that the preferred embodiments may be altered or amended without departing from the true spirit and scope of the disclosure, as defined in the accompanying claims.

What is claimed is:

1. A method for preventing infection associated with implantation of a medical device, comprising:
introducing minocycline microparticles and rifampin microparticles into a solution comprising water to generate an injectable antimicrobial composition, wherein the minocycline and rifampin microparticles are introduced into the solution during a procedure for implanting a medical device in a patient; and
administering the injectable antimicrobial composition to the patient in proximity to the medical device.

2. The method of claim 1, further comprising placing the medical device in a surgical pocket of the patient, wherein administering the injectable antimicrobial composition to the patient in proximity to the medical device comprises administering the antimicrobial composition to the surgical pocket.

3. The method of claim 1, wherein the minocycline microparticles are biodegradable and wherein the rifampin microparticles are biodegradable.

4. The method of claim 1, wherein the minocycline microparticles comprise poly(lactic-co-glycolic acid) and wherein the rifampin microparticles comprise poly(lactic-co-glycolic acid).

5. A kit comprising:
a first container containing minocycline microparticles, wherein the minocycline microparticles comprise poly(lactic-co-glycolic acid) and minocycline; and
a second container containing rifampin microparticles, wherein the rifampin microparticles comprise poly(lactic-co-glycolic acid) and rifampin.

6. The kit of claim 5, further comprising a third container for combining the minocycline microparticles and the rifampin microparticles, the third container being empty prior to addition of the minocycline microparticles and the rifampin microparticles.

7. The kit of claim 6, wherein the third container includes markings indicating the desired volumes of minocycline microparticles and rifampin microparticles.

8. The kit of claim 7, wherein the third container further includes a marking indicating the desired volume of an aqueous solution to be added to the minocycline and rifampin microparticles.

9. The kit of claim 5, wherein the first container includes sufficient empty space, when containing the minocycline microparticles, for addition of a sufficient volume of the rifampin microparticles to obtain a desired ratio of minocycline microparticles to rifampin microparticles, or wherein second container includes sufficient empty space, when containing the rifampin microparticles, for addition of a sufficient volume of the minocycline microparticles to obtain a desired ratio of minocycline microparticles to rifampin microparticles.

10. The kit of claim 9, wherein the first container includes a marking indicating a level to which the rifampin microparticles should be added to obtain the desired ratio of minocycline microparticles to rifampin microparticles, or wherein the second container includes a marking indicating a level to which the minocycline microparticles should be added to obtain the desired ratio of rifampin microparticles to minocycline microparticles.

11. The kit of claim 9, wherein the first container includes sufficient empty space, when containing the minocycline microparticles and the desired ratio of rifampin microparticles, to add a sufficient amount of a solution to suspend the minocycline microparticles and the desired ratio of rifampin microparticles in a desired volume, or wherein the second container includes sufficient empty space, when containing the rifampin microparticles and the desired ratio of minocycline microparticles, to add a sufficient amount of a solution to suspend the rifampin microparticles and the desired ratio of minocycline microparticles in a desired volume.

12. The kit of claim 11, wherein the first container includes a first marking indicating a level to which the rifampin microparticles should be added to obtain the desired ratio of minocycline microparticles to rifampin microparticles and includes a second marking indicating the level to which the solution should be added to obtain the desired volume of suspended microparticles, or wherein the second container includes a first marking indicating a level to which the minocycline microparticles should be added to obtain the desired ratio of rifampin microparticles to minocycline microparticles and includes a second marking indicating the level to which the solution should be added to obtain the desired volume of suspended microparticles.

13. The kit of claim 5, further comprising a solvent container housing an aqueous solution for suspending the minocycline and rifampin microparticles.

14. The kit of claim 13, further comprising a syringe for injecting suspended minocycline and rifampin microparticles into a patient.

15. The kit of claim 5, further comprising a syringe for injecting suspended minocycline and rifampin microparticles into a patient.

16. The kit of claim 5, wherein the amount of minocycline microparticles in the first container and the amount of rifampin microparticles in the second container are configured such that together, the entire contents of the first and second container, provide minocycline and rifampin in a releasable amount sufficient to treat an infection associated with implantation of a medical device in a patient.

17. A microparticle for releasing therapeutic agents into a patient, comprising
a biodegradable polymer;
minocycline; and
rifampin.

18. The microparticle of claim 17, wherein the biodegradable polymer comprises poly(lactic-co-glycolic acid).

19. A method for preventing infection associated with implantation of a medical device, comprising:
introducing a plurality of microparticles, wherein each microparticle comprises minocycline and rifampin, into a solution comprising water to generate an injectable antimicrobial composition, wherein the microparticles are introduced into the solution after placement of a medical device a patient; and
administering the injectable antimicrobial composition to the patient in proximity to the medical device.

20. The method of claim 19, further comprising placing the medical device in a surgical pocket of the patient, wherein administering the injectable antimicrobial composition to the patient in proximity to the medical device comprises administering the antimicrobial composition to the surgical pocket.
